(19) United States
(12) Patent Application Publication
(10) Pub. No.: US 2007/0021672 A1
(43) Pub. Date: Jan. 25, 2007

(54) Cuff for blood pressure monitor

(75) Inventors: Jong Youn Lee, Yongin-si (KR); Kyung Ho Kim, Yongin-si (KR); Eun Young Choe, Suwon-si (KR); Hye Jin Jung, Seoul (KR)

Correspondence Address: STAAS & HALSEY LLP, SUITE 700, 1201 NEW YORK AVENUE, N.W., WASHINGTON, DC, 20005, US

(73) Assignee: Samsung Electronics Co., LTD., Suwon-si (KR)

(21) Appl. No.: 11/418,258

(22) Filed: May 5, 2006

(30) Foreign Application Priority Data
Jul. 20, 2005 (KR) 10-2005-0066042

(51) Int. Cl. A61B 5/02

(52) U.S. Cl. 600/499; 600/485; 600/490

(57) ABSTRACT

A cuff of a blood pressure monitor includes: a pressing part surrounding a first finger and pressing against the first finger by using air pressure; a cuff housing covering an outer circumference of the pressing part; and a cuff guide formed on a side surface of the cuff housing and in contact with a second finger adjacent to the first finger to guide positioning of the cuff housing. The cuff guide is formed corresponding to a shape of the second finger and maintains a desired positioning of the cuff housing, thus constraining insertion of the first finger at a predetermined angle. Accordingly, a position of an artery of the first finger inserted into the cuff with respect to a blood pressure sensing portion improves the accuracy of measuring the blood pressure.

### CROSS-REFERENCE TO RELATED APPLICATIONS

This application claims the benefit of Korean Patent Application No. 2005-66042, filed on Jul. 20, 2005, in the Korean Intellectual Property Office, the disclosure of which is incorporated herein in its entirety by reference.

### BACKGROUND OF THE INVENTION

1. Field of the Invention

The present invention relates to a cuff for a blood pressure monitor for a finger, and more particularly, to a cuff for a blood pressure monitor which improves the sensitivity with respect to blood pressure measurement, and is also convenient to use.

2. Description of the Related Art

FIG. 1 is a perspective view illustrating a conventional a blood pressure monitor for a finger.

Referring to FIG. 1, a conventional blood pressure monitor includes a main unit 10 and a cuff unit 20, and the main unit 10 is connected to the cuff unit by a cable 15. A display unit 24 is provided on the top of the cuff unit 20, in which a cuff for pressing against a finger by using compressed air is provided.

A user may operate the blood pressure monitor by inserting a finger into the cuff 22. The cuff 22 of the cuff unit 20 may be expanded by compressed air while in contact with the finger and perform a process of raising the pressure and a process of dropping the pressure of the cuff. In this case, a photoelectric sensor is installed on the surface of the cuff 22 to measure blood pressure according to the amount of light transmitted.

FIG. 2 is a cross-sectional view illustrating a state in which blood pressure is correctly measured in the blood pressure monitor of FIG. 1, and FIG. 3 is a cross-sectional view illustrating a state in which blood pressure is incorrectly measured in the blood pressure monitor of FIG. 1.

Referring to FIG. 2, a user may insert a finger into the cuff 22 and operate the blood pressure monitor so that the finger is in direct contact with the cuff due to expansion of the cuff. A light-emitting device 32 is installed on the inward facing surface of the cuff 22 and may emit light toward an artery 1, and a light-receiving device 34 is installed adjacent to the light-emitting device 32 to sense the reflected light. In a conventional blood pressure monitor, a pulse wave measured from the photoelectric sensor may be sensed while the pressure is increased or decreased, and the characteristic of the blood pressure may be computed from the change of the measured pulse wave. The computed blood pressure is displayed outside by a display unit 24 shown in FIG. 1.

However, in order to measure the blood pressure of the user precisely, the artery of the finger is located adjacent to the photoelectric sensor. If the position of the finger is changed so that the location of the artery is changed, a blood pressure index cannot be measured or may be inaccurately measured.

Referring to FIG. 3, a finger may rotate while being inserted into the cuff. The location of the artery 1 is relatively far from the light-emitting device 32 and the light-receiving device 34. Therefore, the light emitted from the light-emitting device 32 may be transmitted to the light-receiving device 34 via a relatively long path. Since the light must travel a longer path, a wave pattern of the light transmitted to the light-receiving device 34 may be changed, thereby resulting in an inaccurate blood pressure index. In addition, in a serious case, it may not be possible to measure the blood pressure.

In the case in which a sound or other transmission means is used instead of the photoelectric sensor, it is also preferable that the artery that is the object to be measured is located adjacent to the blood pressure sensor. However, in the conventional cuff for a blood pressure monitor, since the position during insertion of the finger may be not consistent, different measured values may be obtained, depending on the positioning of the finger, even under the same conditions.

### SUMMARY OF THE INVENTION

The present invention provides a cuff for a blood pressure monitor, which can naturally guide a sensor for sensing blood pressure so that the sensor is located adjacent to an artery.

The present invention also provides a cuff for a blood pressure monitor, which can guide positioning and locating of the blood pressure monitor to improve sensitivity and accuracy with respect to blood pressure measurement.

The present invention also provides a cuff for a blood pressure monitor, which can prevent a folded portion adjacent to a chamber from being expanded and exposed outside the chamber while an expansion portion of the blood pressure monitor is expanded.

According to an aspect of the present invention, a cuff guide is formed on the side surface of a cuff housing to keep a predetermined angle and a predetermined position of installing a cuff for a blood pressure monitor. To dispose a pressing part, including a tube for the cuff or an expansion portion around a certain finger, in an accurate position, the cuff guide may use another finger adjacent to the finger in which the blood pressure is being measured. Specifically, the cuff guide is formed in contact with an adjacent finger on the side surface of the cuff housing, thus disposing the pressing part contained in the cuff housing in a desired position.

To form the cuff guide in the cuff housing, a concave or embaying portion may be formed in a single body with the cuff housing. In addition, the cuff guide may be manufactured separately and then attached or fixed to the cuff housing, thus adding guide function.

If the location and the position of the cuff housing can be constantly maintained by using the cuff guide around the finger, a blood pressure sensing part may be installed adjacent to an artery of the finger based on the fixed cuff housing. In this case, the blood pressure sensing part may be installed on the surface of the inside of a pressing part, which may be variously controlled according to the sensing characteristic of the blood pressure sensing part. For example, a blood pressure sensing part, such as a photoelectric sensor, may be installed on the inner surface of the pressing part, or a blood pressure sensing part, such as a microphone, may be installed inside the pressing part.

A cuff for a blood pressure monitor generally includes a pressing part and a cuff housing, and the pressing part includes an expansion portion to press against a finger by using compressed air. In the present invention, since a folded portion is formed around the expansion portion, a surface of an air chamber of the expansion portion, which is in contact with the finger, may have a substantially constant length while the expansion portion expands.

A side covering portion is formed at the end portions of the cuff housing, adjacent to a peak of the folded portion. The side covering portion may cover the peak of the folded portion when the expansion portion is expanding by air pressure, thus preventing the folded portion from unexpectedly expanding outside of the cuff housing.

Additional aspects and/or advantages of the invention will be set forth in part in the description which follows and, in part, will be apparent from the description, or may be learned by practice of the invention.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is a perspective view illustrating a conventional blood pressure monitor for a finger;

FIG. 2 is a cross-sectional view illustrating an example in which blood pressure is normally measured in the blood pressure monitor of FIG. 1;

FIG. 3 is a cross-sectional view illustrating an example in which blood pressure is abnormally measured in the blood pressure monitor of FIG. 1;

FIG. 4 is a perspective view of a cuff for a blood pressure monitor, according to an embodiment of the present invention;

FIG. 5 is a front view of the cuff of FIG. 4;

FIG. 6 is a perspective view illustrating an example of using the cuff of FIG. 4;

FIG. 7 is a cross-sectional view illustrating the cuff of FIG. 4;

FIG. 8 is an exploded perspective view of a cuff for a blood pressure monitor, according to another embodiment of the present invention; and

FIG. 9 is a cross-sectional view of the cuff of FIG. 8.

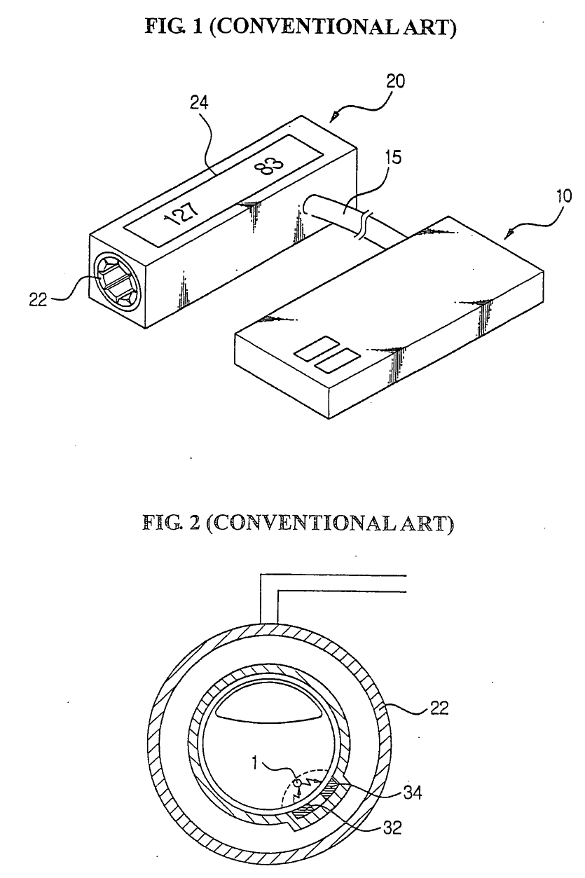

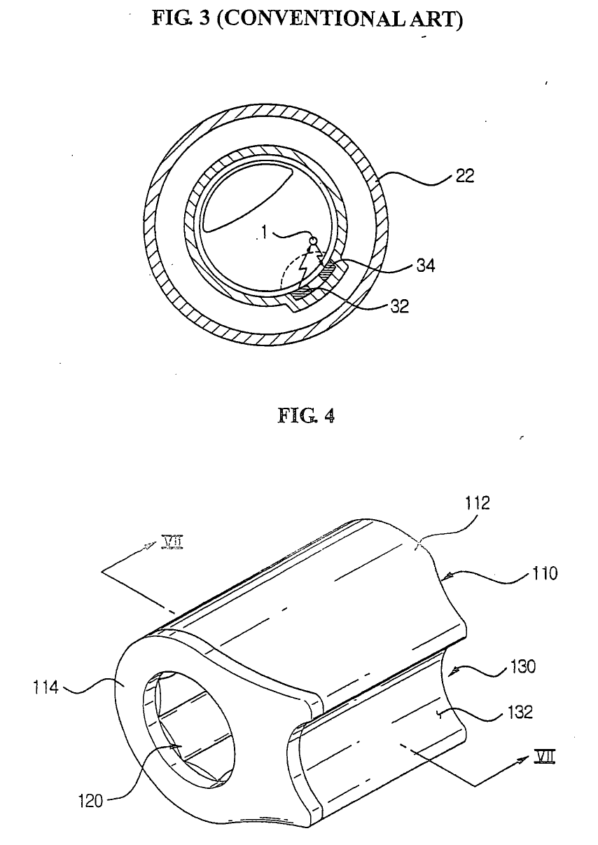

### DETAILED DESCRIPTION OF THE PREFERRED EMBODIMENTS

Reference will now be made in detail to the embodiments of the present invention, examples of which are illustrated in the accompanying drawings, wherein like reference numerals refer to the like elements throughout. The embodiments are described below to explain the present invention by referring to the figures.

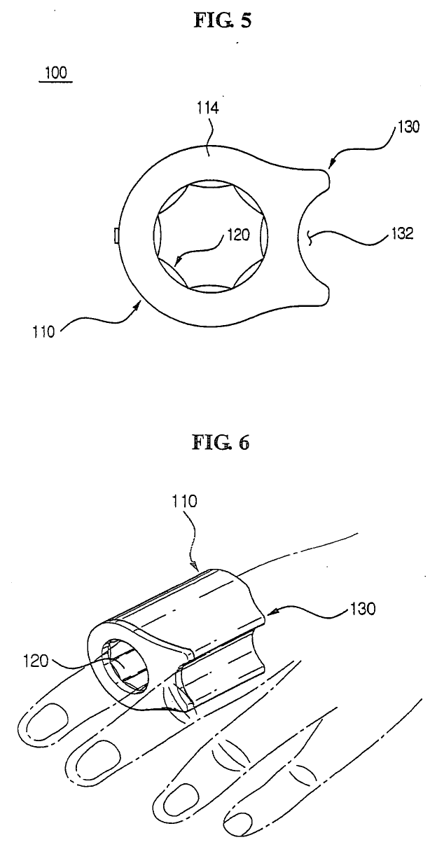

FIG. 4 is a perspective view of a cuff for a blood pressure monitor, according to an embodiment of the present invention, FIG. 5 is a front view of the cuff of FIG. 4, and FIG. 6 is a perspective view illustrating an example of using the cuff of FIG. 4.

Referring to FIGS. 4 and 5, a cuff 100 for a blood pressure monitor includes a cuff housing 110, a pressing part 120, and a cuff guide 130. The pressing part 120 is installed in the cuff housing 110 and disposed along the inner circumference of the cuff housing 110. Since the cuff housing 110 has an approximately cylindrical inner wall, the pressing part installed inside the cuff housing 110 may provide a cylindrical path through the cuff 100.

Though the cuff 100 itself may contain all the necessary elements to measure blood pressure independently, the cuff may be constructed to measure blood pressure via a functional connection with an external pump or a control unit. In the latter case, a user inserts a finger into the cuff 100 and may operate the cuff 100 by using an internal or external control unit.

In the operation of the cuff 100, the pressing part 120 is expanded to be in close contact with an inserted finger, and as the air pressure increases, presses against the circumference of the finger to contact the finger directly. Also, after increasing beyond a certain predetermined pressure, the pressure inside the pressing part 120 is reduced. In a procedure of reducing the pressure after the increase in pressure, a blood pressure sensing part installed in the pressing part 120 may sense a change of an artery via a photoelectric sensor or a sonic sensor, and a control unit may compute the blood pressure of a user based on the sensed data.

The cuff 100 according to an embodiment of the present invention uses a photoelectric sensor as a sensor. The cuff 100 may measure a change in the pulse wave via the photoelectric sensor installed in the pressing part 120 while the pressure changes, and may compute the blood pressure of the user from the change of the pulse wave.

An artery of a finger is disposed according to both side surfaces of the finger. Accordingly, when the user inserts a finger into a cuff for a blood pressure monitor, if the finger is positioned incorrectly, the artery of the finger is distanced from the blood pressure sensor. The larger the distance between the blood pressure sensor and the artery, the less accurate the measurement and the sensitivity become with respect to the blood pressure.

As shown in FIGS. 4 and 5, the cuff 100 according to the present embodiment includes a cuff guide 130, which may prevent the finger of the user from being positioned improperly while being inserted into the cuff by using the cuff guide 130.

Described in detail, the cuff guide 130 is located along the side surface of the cuff housing 110. The cuff guide 130 is formed as a single body with the cuff housing 110 via injection molding or compression molding. The cuff guide 130 protrudes on a sidewall of the cuff housing 110 in the shape of a letter “M,” and the center portion of the cuff guide 130 is a curved surface, thus comfortably receiving the adjacent finger. The “M” shaped cuff guide may also be described as a concave or embaying portion, and may be formed in a single body with the cuff housing or may be manufactured separately and then attached or fixed to the cuff housing, thus adding guide function.

The cuff housing 110 includes a housing body 112 and a housing cover 114 and contains the pressing part 120 by using a space provided inside the housing body 112 and the housing cover 114. The housing body 112 is formed as a single body with the cuff guide 130 to provide a concave portion 132 to position the cuff 100 using an adjacent finger.

Referring to FIGS. 5 and 6, the cuff 100 is placed on the index finger of the left hand and encircles the index finger as a ring. A concave portion 132 is formed in the center portion of the cuff guide 130, and the concave portion 132 is formed approximately corresponding to the shape of an adjacent finger to position the index finger. Accordingly, as illustrated, a user inserts the index finger into the cuff 100 and controls the positioning so that the cuff housing 110 is fixed in a certain predetermined position. Since the index finger and the middle finger are side by side, the cuff 100 may contain the index finger in an approximately horizontal posture. Though the shape and the disposition of the fingers are different depending upon the users, this is a small difference, and the cuff 100 may contain a finger in a substantially same posture regardless of the users and may easily estimate the position of an artery in the inserted finger.

Therefore, the artery may be disposed in approximately the same position relative to the blood pressure sensing part in the cuff 100 by using the cuff guide 130.

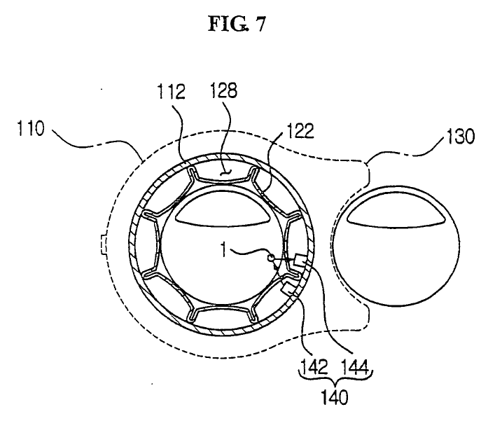

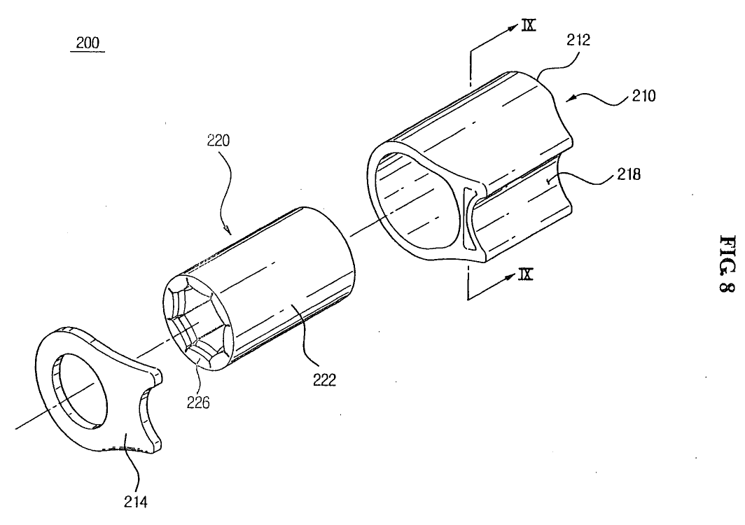

FIG. 7 is a cross-sectional view illustrating the cuff of FIG. 4. For reference, FIG. 7 illustrates the cuff 100 based on the line of VII-VII, and an index finger and a middle finger are illustrated for explaining the position between the blood pressure sensing unit and the fingers.

Referring to FIG. 7, the cuff guide 130, in the shape of an “M” in the present embodiment, includes two projection portions in contact with the upper portion and the lower portion of the side surface of the middle finger. On the other hand, according to another embodiment, the cuff guide 130 may include one projection portion and be in contact with the upper portion or the lower portion of the side surface of the middle finger by using the one projection. In the case of using one projection portion, it may be difficult to maintain contact of the cuff with an adjacent finger. However, since the cuff 100 may rotate on the inserted finger, the position of the cuff may be adjusted by rotating the cuff after inserting the finger into the cuff.

Also, as illustrated, a blood pressure sensing portion 140 is installed in the pressing part 120. The blood pressure sensing portion 140 includes a light-emitting device 142 and a light-receiving device 144, and the light-emitting device 142 is located adjacent to the light-receiving device 144. When a user inserts an index finger into the pressing part 120 in the cuff housing 110 and supports the cuff guide 130 by a middle finger, the cuff housing 110 and the fingers are located side by side, and the blood pressure sensing portion 140 may be automatically located adjacent to the artery 1 inside the index finger. In the present embodiment, the light-emitting device 142 and the light-receiving device 144 are located inside the expansion portion 122. According to another embodiment of the present invention, a light-emitting device and a light-receiving device may be located on the surface of the expansion portion 122. Also, in the present embodiment, the light-emitting device 142 is located adjacent to the light-receiving device 144. A different configuration may have a light-emitting device located opposite to a light-receiving device and light penetrates a finger and may transmit information on blood pressure.

Of course, various kinds of conventional sensors for measuring blood pressure, such as a microphone, may be used in addition to, or as a substitution for, a photoelectric sensor. The sensors may be disposed in a desired position corresponding to the position of a predetermined artery.

Referring to FIG. 7, the pressing part 120 includes the expansion portion 122. The expansion portion 122 is formed along the inner surface of the cuff housing 110, in which a plurality of air chambers 128 may be formed, because a folded portion is formed at a predetermined interval. As described above, the expansion portion 122 is formed in a section shape instead of a tube shape, which cannot form a sealed space by itself. The expansion portion 122 may form the sealed air chambers 128 only by combining with the cuff housing 110 to be sealed. Namely, the expansion portion 122 is located inside the air chamber 128 to define the inward facing surface of the air chamber 128, and the inner wall of the cuff housing 110 is located outside the air chamber 128 to define the outward facing surface of the air chamber 128.

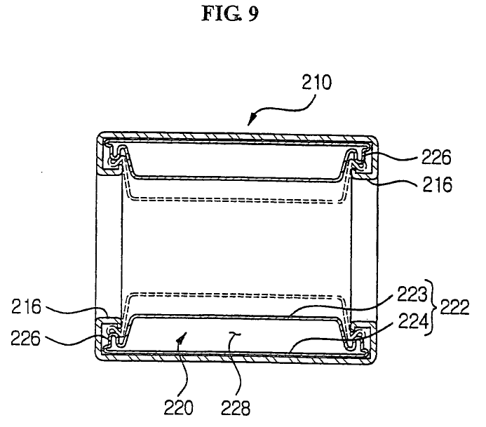

FIG. 8 is an exploded perspective view of a cuff for a blood pressure monitor, according to another embodiment of the present invention, and FIG. 9 is a cross-sectional view of the cuff of FIG. 8. For reference, FIG. 9 illustrates a section made by cutting a cuff 200 for a blood pressure monitor based on IX-IX, and the cuff 200 of the present embodiment may be explained by referring to the description and drawings of the previous embodiment.

Referring to FIGS. 8 and 9, the cuff 200 includes a cuff housing 210 and a pressing part 220. The pressing part 220 is installed inside the cuff housing 210, is disposed along the circumference of the cuff housing 210, and may provide a cylindrical path inside the cuff 200.

The cuff 200 may independently measure blood pressure by itself or may measure blood pressure via a functional connection with an external pump or a control unit. When the cuff 200 operates, the pressing part 220 is expanded to contact a finger inserted into the cuff, and the air pressure is increased so that the pressing part 220 contacts the finger directly around the circumference of the finger. After the pressure is increased beyond a predetermined point, the pressure inside the pressing part 220 is reduced. In the process of increasing and decreasing the pressure, a blood pressure sensing part may sense a change in an artery via a photoelectric sensor or a sonic sensor, and a control unit may compute the blood pressure of a user based on sensed changes of the artery. The cuff 200, according to the present embodiment, uses a photoelectric sensor as a sensor, measures a pulse wave changed according to the changed pressure via the photoelectric sensor installed in the pressing part 220, and computes the blood pressure of the user from the change of the changed pulse wave.

The cuff housing 210 includes a housing body 212, a housing cover 214, a concave portion 218 and contains the pressing part 220 by using a space provided inside the housing body 212 and the housing cover 214. The concave portion 218 is formed as a single body on the side surface of the housing body 212 and may be manufactured with the housing body 212 as one piece by injection molding. In the present embodiment, the concave portion 218 is formed in the shape of an “M,” and may prevent a finger of a user from being positioned improperly while the finger is inserted by using a depression portion in the middle of the concave portion 218.

Since the center of the concave portion 218 is concave in shape, another adjacent finger is comfortably disposed. For example, in the case the cuff 200 is put on the index finger of the left hand and encircles the index finger as a ring, the concave portion is formed corresponding to the adjacent middle finger to position the middle finger, and thus also position the index finger with respect to the blood pressure sensing part (not shown). Accordingly, a user inserts the index finger into the cuff 200 and controls the position by contacting the adjacent middle finger of the same hand with the concave portion 218, thus positioning the cuff housing 210 in a predetermined position. Though the shape of a hand and the disposition of fingers are different for different users, it is only a small difference. The cuff 200 may contain a finger in substantially the same position regardless of the user, and may easily estimate the position of the artery of the inserted finger. The artery may be located in approximately the same position in the cuff 200 by using the concave portion 218, and the blood pressure sensing part is installed adjacent to an expected position of the artery, thus improving the accuracy of sensing blood pressure.

According to FIGS. 8 and 9, the pressing part 220 includes an expansion portion 222, in the shape of a tube, and a folded portion 226, formed in both ends of an air chamber 228. The expansion portion 222 is located along the inner surface of the cuff housing 210, and includes the air chamber 228 defined by an inner surface 223 and an outer surface 224. Since the inner surface 223 of the expansion portion 222 forms a portion folded at a certain interval, a plurality of the air chambers 228 is formed. The outer surface 224 of the expansion portion 222 is provided along the inner surface of the cuff housing 210 to define an external boundary of the air chamber 228. In the present embodiment, the expansion portion 222 is formed in the shape of a tube having two layers and may form a sealed space for itself. The plurality of the air chambers 228 is disposed in a circumferential direction, and the folded portion 226 is provided adjacent to the front/rear ends of each of the air chambers 228. The folded portion 226 may include at least one wrinkle. When compressed air is provided and the volume of the air chamber 228 is increased, the folded portion 226 is partially unfolded or rotated, and the innermost surface of the air chamber 228 maintains the same length while being expanded.

As illustrated in FIG. 9, the air chamber 228 is expanded, the folded portion 226 is unfolded, and the position is entirely moved up. However, a side covering portion is provided at the both ends of the cuff housing 210, and covers a peak of the folded portion 226, thus preventing the expanded folded portion 226 from instantly expanding outside of the cuff housing 210. Since the side covering portion 216 is curved toward the inside, it induces the folded portion 226 to grow toward the inside the side covering portion 216, and the folded portion 226 is preliminarily prevented from being exposed outside the housing body of the cuff.

A blood pressure sensing part is installed in the pressing part 220. The blood pressure sensing part may include a light-emitting device and a light-receiving device and estimate the position of an artery of an inserted finger, thus being installed corresponding to the predicted position of the artery.

The cuff according to the present invention corrects the position and posture of a housing by using a cuff guide or a concave portion to prevent a finger from being positioned improperly while the finger is inserted into the cuff. Namely, the finger is constrained to be inserted at a predetermined angle, thus easily estimating the position of an artery of the finger and installing a blood pressure sensing part adjacent to the artery.

As a result, the position of the finger in which the blood pressure is to be measured and the cuff for a blood pressure monitor are guided, thus installing the blood pressure sensing part adjacent to the artery of the finger in which the blood pressure is to be measured, improving the sensitivity and accuracy of measuring the blood pressure by using light or sound.

Also, in the case of a conventional blood pressure monitor, it may be possible that a folded portion is exposed outside or instantly expanded while a tube of a blood pressure monitor is expanded. However, in the present invention, the expansion of a folded portion may be guided by using a side covering portion covering the folded portion to prevent the folded portion from being exposed outside of the cuff or from being instantly expanded.

Although a few embodiments of the present invention have been shown and described, it would be appreciated by those skilled in the art that changes may be made in these embodiments without departing from the principles and spirit of the invention, the scope of which is defined in the claims and their equivalents.

What is claimed is:

1. A cuff of a blood pressure monitor, comprising:
a pressing part surrounding a first finger and pressing against the first finger by using air pressure;
a cuff housing covering an outer circumference of the pressing part; and
a cuff guide formed on a side surface of the cuff housing and in contact with a second finger adjacent to the first finger to guide positioning of the cuff housing.

2. The cuff of claim 1, wherein the cuff guide is formed in a single body with the cuff housing and is formed in a concave shape that corresponds to the shape of the second finger.

3. The cuff of claim 1, wherein the pressing part includes a tube expansion portion to contain compressed air, in which an inner surface of the tube expansion portion is expanded to press against the first finger, and the outer surface of the tube expansion portion defines at least one air chamber with the inner surface.

4. The cuff of claim 1, wherein the pressing part includes a single-layer expansion portion to contain compressed air together with an inner surface of the cuff housing, in which the single-layer expansion portion is expanded to press against the first finger and defines at least one air chamber together with the inner surface of the cuff housing.

5. The cuff of claim 1, further comprising a blood pressure sensing part installed in the pressing part to measure blood pressure, wherein the blood pressure sensing part is installed adjacent to an artery of the first finger and the cuff guide is in contact with the second finger.

6. The cuff of claim 5, wherein the blood pressure sensing part includes a light-emitting device disposed adjacent to the artery of the first finger and a light-receiving device formed adjacent to the light-emitting device to receive light reflected by the artery when the cuff guide is in contact with the second finger.

7. A cuff of a blood pressure monitor, comprising:
a pressing part surrounding a first finger and pressing against the first finger by using air pressure;
a cuff housing covering an outer circumference of the pressing part and including a concave portion to contain, partially, a second finger adjacent to the first finger; and
a blood pressure sensing part installed in the pressing part to be positioned adjacent to an artery of the first finger when the second finger is contained in the concave portion.

8. The cuff of claim 1, wherein the pressing part includes a tube expansion portion to contain compressed air, in which an inner surface of the tube expansion portion is expanded to press against the first finger and an outer surface of the tube expansion portion defines a plurality of air chambers together with the inner surface of the tube expansion portion.

9. The cuff of claim 1, wherein the pressing part includes a single-layer expansion portion to contain compressed air together with an inner surface of the cuff housing, in which the single-layer expansion portion is expanded to press against the first finger and defines a plurality of air chambers together with the inner surface of the cuff housing.

10. A cuff of a blood pressure monitor, comprising:
a pressing part including an expansion portion surrounding a first finger to press against the first finger by using air pressure and a folded portion formed at both ends of the expansion portion; and
a cuff housing covering an outer circumference of the pressing part and including a side covering portion covering a peak of the folded portion.

11. The cuff of claim 10, wherein the expansion portion is formed in the shape of a tube and includes an inner surface and an outer surface, in which the inner surface of the expansion portion is expanded to press against the finger, and the outer surface of the expansion portion defines an air chamber together with the inner surface thereof.

12. The cuff of claim 10, wherein the expansion portion is open toward the outer surface, and the inner surface of the expansion portion is expanded to press against the first finger and defines an air chamber together with an inner surface of the cuff housing.

13. The cuff of claim 12, wherein the expansion portion includes a plurality of air chambers formed around the first finger and the folded portion is formed at both ends of the air chamber, respectively.

14. A cuff of a blood pressure monitor, comprising:
a pressing part including an expansion portion defining a plurality of air chambers, the air chambers surrounding a first finger for pressing against the first finger by using air pressure; and
a folded portion formed in a single body with the expansion portion and formed adjacent to both ends of each of the air chambers;
a cuff housing covering an outer circumference of the pressing part and including an embaying portion to contain, partially, a second finger adjacent to the first finger and a side covering portion covering a peak of the folded portion; and
a blood pressure sensing part installed in the pressing part, wherein, upon insertion of the first finger into the cuff, the blood pressure sensing part is located adjacent to an artery of the first finger and the second finger is contained, at least partially, in the embaying portion.

15. The cuff of claim 14, wherein the expansion portion is formed in the shape of a tube and includes an inner surface and an outer surface, in which the inner surface of the expansion portion is expanded to press against the first finger and the outer surface of the expansion portion defines the plurality of air chambers together with the inner surface of the expansion portion.

16. The cuff of claim 15, wherein the expansion portion is open toward the outer surface, in which the inner surface of the expansion portion is expanded to press against the first finger and defines. the plurality of air chambers together with an inner surface of the cuff housing.
